(19) United States
(12) Patent Application Publication
(10) Pub. No.: US 2003/0163082 A1
(43) Pub. Date: Aug. 28, 2003

(54) Lumen weld

(76) Inventor: Steven P. Mertens, New Hope, MN (US)

Correspondence Address: VIDAS, ARRETT & STEINKRAUS, P.A., 6109 BLUE CIRCLE DRIVE, SUITE 2000, MINNETONKA, MN, 55343-9185, US

(21) Appl. No.: 10/082,869

(22) Filed: Feb. 26, 2002

(51) Int. Cl. A61M 3/00

(52) U.S. Cl. 604/43

(57) ABSTRACT

A multi-lumen tubular member having a predetermined length wherein the tubular member comprises an outer tube and at least one inner tube and a shared engagement area. The outer tube is substantially disposed about the at least one inner tube. The at least one inner tube has an outer surface and the outer tube has an inner surface. A portion of the outer surface of the at least one inner tube is engaged to a portion of the inner surface of the outer tube to define the engagement area. The engagement area extends in a substantially longitudinal manner along a substantial portion of the predetermined length. The tubular member is preferably a catheter.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

Catheters having multiple lumens are well known. Multi-lumen catheters are known to have use in infusion, perfusion, hemodialysis and other procedures. Duallumen catheters used for hemodialysis, for example, provide one lumen for arterial (intake) flow of blood to be purified and one lumen for venous (return) flow of purified blood.

In most cases a multi-lumen catheters may be used as or in the same procedure as other catheter types. For example, a multi-lumen catheter may be suitable for use in PTCA procedures. In typical PTCA procedures, a guiding catheter is percutaneously introduced into the cardiovascular system of a patient through a vessel and advanced through therein until the distal end thereof is at a desired location in the vasculature. A guidewire and a dilatation catheter having a balloon on the distal end thereof are introduced through the guiding catheter with the guidewire sliding through the dilatation catheter. The guidewire is first advanced out of the guiding catheter into the patient's coronary vasculature and the dilatation catheter is advanced over the previously advanced guidewire until the dilatation balloon is properly positioned across the lesion. Once in position across the lesion, the flexible, expandable, preformed balloon is inflated to a predetermined size with a liquid or gas at relatively high pressures, to radially compress the arthrosclerotic plaque of the lesion against the inside of the artery wall and thereby dilate the lumen of the artery. The balloon is then deflated to a small profile so that the dilatation catheter may be withdrawn from the patients vasculature and blood flow resumed through the dilated artery.

In the above example the balloon catheter may include a guide wire lumen and an inflation lumen, and may also include one or more additional lumens. In another example a multi-lumen catheter may be suitable for the deliver of implantable medical devices such as stents, grafts, stent-grafts and vena cava filters. The various types of dual-lumen catheters may include a variety of cross-sectional configurations such as for example: two semi-circular lumens arranged in a side-by-side configuration and formed within a unitary tube which is internally divided by a planar septum; two circular lumens arranged in a side-by-side configuration and formed within a unitary tube separated by a septum; circular and crescent-shaped lumens (or circle-C) arranged in a side-by-side configuration and formed within a unitary tube which is divided internally by a curved septum or wall by attaching the exterior surface of an inner tube along its length to the interior surface of an outer tube; elliptically-shaped and circular lumens arranged in a side-by-side configuration and formed within a unitary tube divided by a curved septum; and circular and annular lumens arranged in a coaxial configuration wherein an inner tube is placed within an outer tube and the lumens are divided by the exterior surface of the inner tube.

Some examples of coaxial, dual-lumen catheters may be found in U.S. Pat. Nos. 4,493,696, 4,037,599 and 5,053,004. Example of catheters having a triple-lumen configuration are shown in U.S. Pat. Nos. 4,072,146; 5,221,255; 5,221,256; 5,167,623; 5,207,648; 5,718,876 and 5,879,499.

In many of the known multi-lumen catheter configurations, the lumens are formed by tubular members which extend from the proximal to the distal end of the catheter, or partially therebetween. In some cases the tubular members engaged together. Where the tubular members are engaged, typically the engagement is at one or both ends of the tubular members and/or include circumferential bonds at specific locations along the length of the catheter.

In some other embodiments a multi-lumen tube is extruded or otherwise formed as a single tube with internal septums, membranes or walls which separate and define the individual lumens. However, in such “multi-lumen extrusion” processes, the extruded tube may include a “web” of extruded material in one or more corners of the extrusion. An example of such webbing is illustrated at reference numeral 12 of the prior art extruded tube 10 shown in PRIOR ART FIG. 1. As may be seen, depending on the configuration of extrusion, the presence of webbing 12 takes up space in one or more of the lumens 14 and/or 16. As a result, the cross-sectional area of the lumens 14 and/or 16 is reduced. Thus in order to compensate for the loss of area caused by the webbing 12 the tube 10 must be provided with a greater diameter in order to maintain the desired luminal area. Where the tube 10 is intended for use as a catheter, this increase in diameter may negatively impact the performance of the catheter in terms of its capability to be inserted into vessels of a particular diameter, and potentially its advancement and trackability performance.

Another potentially negative aspect to the presence of webbing is an effect known as “back boning”. The clinical effect of back boning is that when an elongate shaft is torqued, the tubular member fails to display a one to one torque response and has the effect of building input which is then subsequently and suddenly released. Back boning is a phenomena which may occur in an unbalanced tube and which may be exacerbated by the presence of webbing. Back boning in a catheter assembly may negatively affect the manipulation and steerage of the catheter as it is advanced through a body vessel.

Accordingly, there is a need in the art for a method for producing a multi-lumen tubular member, such as a catheter, that avoids the formation of webbing and its potentially negative attributes.

### BRIEF SUMMARY OF THE INVENTION

The present invention is directed to several embodiments. In at least one embodiment, the invention is directed methods for forming and preparing multi-lumen tubular members. In at least one embodiment, the invention is directed to a methods of welding or otherwise engaging two or more tubular members to one another. In at least one embodiment, the tubular members define one or more lumens of a catheter assembly.

In at least one embodiment, the tubular members may be engaged together along a shared longitudinal surface where one tubular member contacts another tubular member. The engagement between members may be along a line which extends continuously or intermittently over the shared length of the tubular members or a portion or portions thereof.

All US patents and applications and all other published documents mentioned anywhere in this application are incorporated herein by reference in their entirety.

The invention in various of its embodiment is summarized below. Additional details of the invention and/or additional embodiments of the invention may be found in the Detailed Description of the Invention below.

The abstract provided herewith is intended to comply with 37 CFR 1.72 and is not intended be used in determining the scope of the claimed invention.

### BRIEF DESCRIPTION OF THE SEVERAL VIEWS OF THE DRAWINGS

A detailed description of the invention is hereafter described with specific reference being made to the following drawings.

FIG. 1 is a PRIOR ART cross-sectional view of an example of a an extruded multi-lumen catheter.

FIG. 2 is a cross-sectional view of an embodiment of the invention.

FIG. 3 is a perspective view of an embodiment of the invention.

FIG. 4 is a perspective view of an embodiment of the invention.

FIG. 5 is a perspective view of an embodiment of the invention.

FIG. 6 is a perspective view of an embodiment of the invention.

FIG. 7 is a cross-sectional view of an embodiment of the invention.

FIG. 8 is a cross-sectional view of an embodiment of the invention.

FIG. 9 is a cross-sectional view of an embodiment of the invention.

FIG. 10 is a cross-sectional view of an embodiment of the invention.

FIG. 11 is a cross-sectional view of an embodiment of the invention.

### DETAILED DESCRIPTION OF THE INVENTION

While this invention may be embodied in many different forms, there are described in detail herein specific preferred embodiments of the invention. This description is an exemplification of the principles of the invention and is not intended to limit the invention to the particular embodiments illustrated.

As indicated above, FIG. 1 illustrates a typical example of a PRIOR ART multi-lumen extrusion tubular member 10. As is shown, such multi-lumen extrusion tubes often include a build up of webbing 12, in one or more of the lumens 14 and 16. It is desirous to provide a multi-lumen tubular member, especially for catheter applications, which avoids the presence of webbing, while continuing to provide for a tubular member having multiple lumens that are longitudinally engaged to one another.

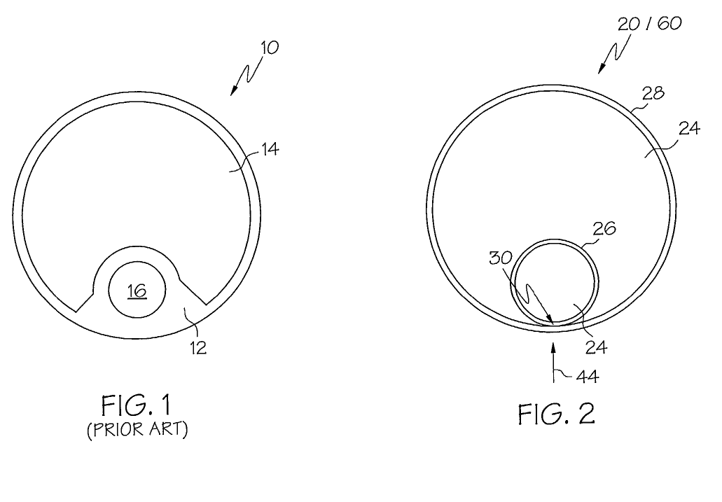

In the embodiment shown in FIG. 2, a dual-lumen tubular member 20 is shown which includes a first or inner lumen 22 and a second or outer lumen 24. Inner lumen 22 is defined by an inner tube or membrane 26. Outer lumen 24 is defined by an outer tube or membrane 28. The inner tube 26 and the outer tube 28 may be constructed from the same or different material(s) as desired. The present embodiment may be characterized as a tube 26 within a tube 28 where in each tube 26 and 28 are distinct members that are engaged along a common engagement area 30. Engagement area 30 may be formed by any known means of engaging materials to one another. Such engagement methods include, but are not limited to physical and chemical bonding such as by the formation of a weld, adhesive bond, mechanical engagement, or other engagement means. Where the engagement area 30 is a weld, the weld is preferably formed through the application of energy indicated by arrow 44. Energy for forming such a weld may be RF, IR, UV, selected frequencies of laser light, ultra-sonic and electrical energy among others.

By providing two separate tubes 26 and 28 which are then bonded, welded or otherwise engaged to one another, the present dual-lumen tubular member 20 avoids the presence of webbing which prior formation techniques such as the multi-lumen extrusion shown in FIG. 1 are known to include.

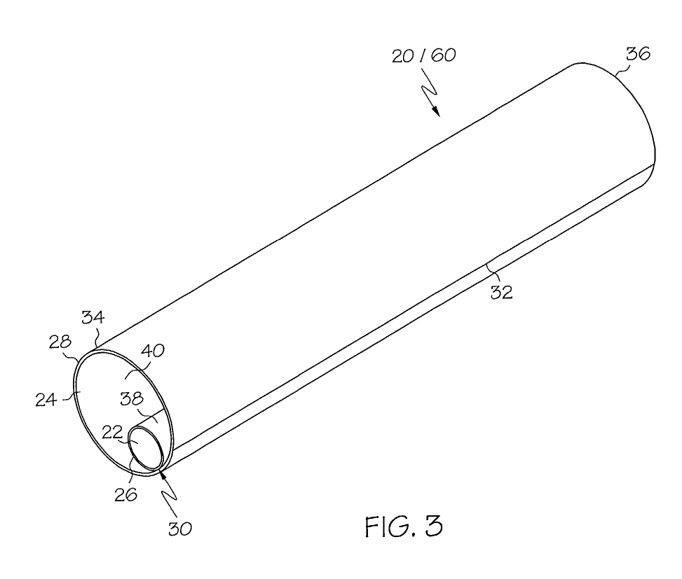

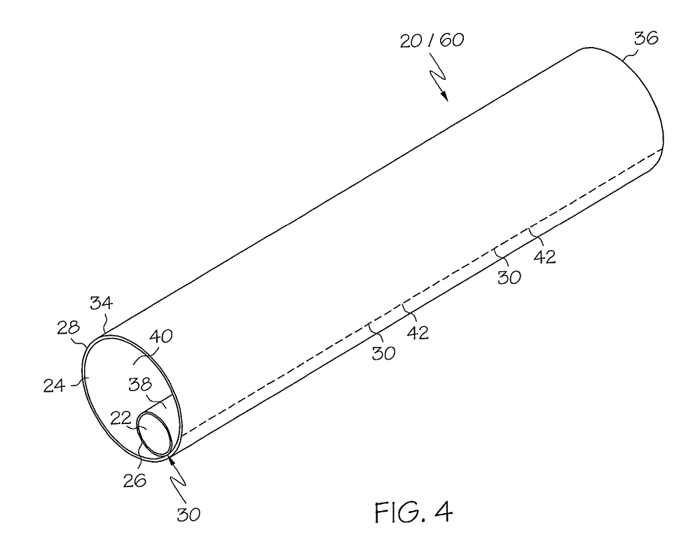

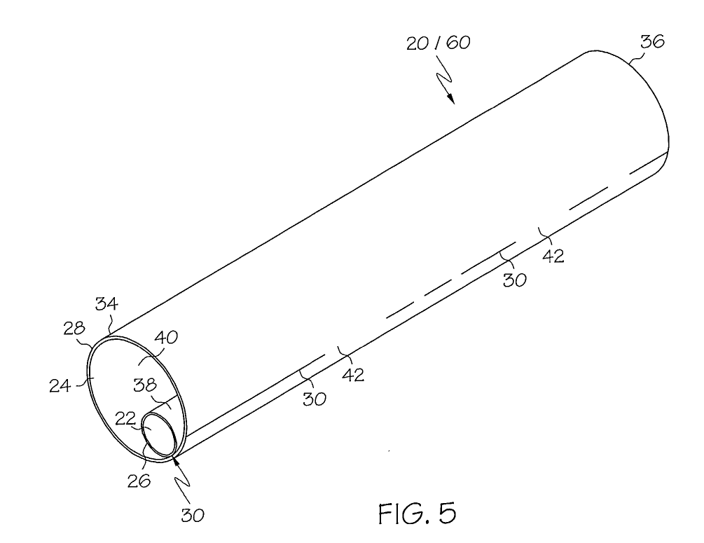

The manner in which inner tube 26 is engaged to outer tube 28 may be varied. In at least one embodiment, shown in FIG. 3, the engagement area 30 extends constantly in a longitudinal “line” 32 from substantially the distal end 34 of the tubular member 20 to the proximal end 36 of the tubular member 20. The longitudinally extending engagement area 30 is the contact point between the outer surface 38 of the inner tube 26 where it engages the inner surface 40 of the outer tube 28. As indicated above, the engagement area 30 may be an uninterrupted line 32, such as is shown in FIG. 3. Alternatively the line 32, may be comprised of a pattern of repeating engaged areas 30 and unengaged areas 42 such as is shown in FIGS. 4 and 5. In the embodiment shown in FIG. 4, the line 32 is a regularly repeating pattern of engaged areas 30 and unengaged areas 42, and in the embodiment shown in FIG. 5 the line 32 is a random, or irregular, pattern of engaged areas 30 and unengaged areas 42. In the various embodiments the engagement area is substantially linear and is not a web or webbing of engaged areas.

Preferably, the engaged area 30 comprises at least 10 percent of the common length of the inner tube 26 and outer tube 28. In some embodiments the engaged area 30 may comprise more than 10 percent of the common length of the inner tube 26 and outer tube 28. In some embodiments the engaged area 30 may be between about 11 to about 25 percent of the common length. In some embodiments engaged area 30 comprises about 50 percent or more of the common length, and in some embodiment the engaged are 30 comprises substantially the entire common length of the inner tube 26 and outer tube 28.

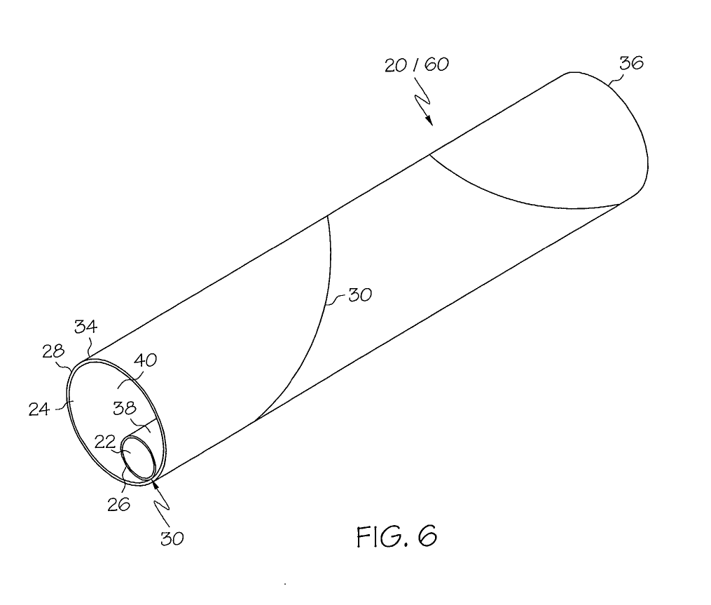

In the various embodiments shown discussed thus far, the inner tube 26 is oriented in a substantially linear manner that is substantially parallel with the outer tube 28. In an alternative embodiment shown in FIG. 6, the inner tube 26 may be helically, or otherwise oriented in a non-linear manner relative to the outer tube 28. In the present embodiment, the inner tube 26 is engaged to the outer tube 28 along a engagement area 30 such as is shown. However, regardless of the orientation of the inner tube 26 to the outer tube 28, the engagement area 30 between the tubes may be continuous, intermittent, or patterned as previously shown and discussed.

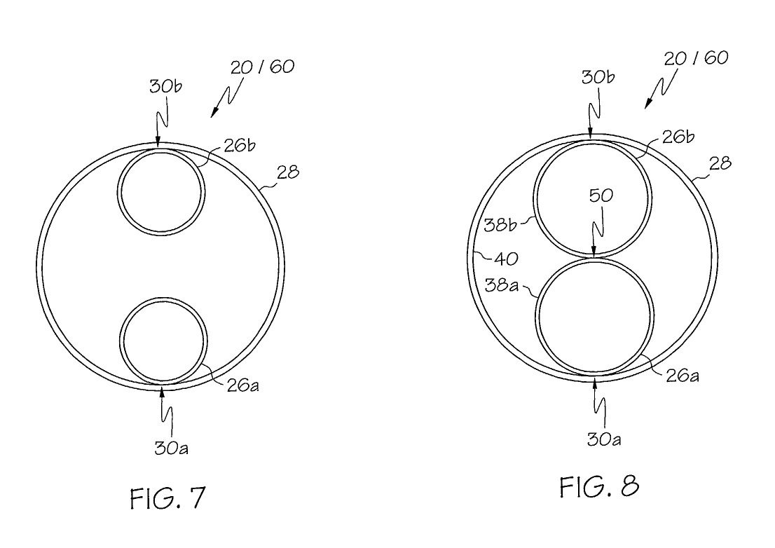

In addition to providing for a variety of bond configurations and tube orientations, the present invention, in some embodiments, is also directed to various arrangements of multi-lumen tubular members which are comprised of more than two tubes. For example, in the embodiment shown in FIG. 7, the tubular member 20 is comprised of an outer tube 28 which contains a first inner tube, 26a as well as a second inner tube 26b. Each inner tube 26a and 26b may be engaged to the outer tube 28 along an engagement area 30a and 30b respectively. Like the previously discussed embodiments above, the engagement areas extend in a substantially longitudinal manner along the substantial length of the tubular member 20. The inner tubes 26a and 26b may be oriented relative to the outer tube 28 in any manner desired. The inner tubes 26a and 26b may also be bonded to the outer tube 28 in any manner desired such as along a continuous, or intermittent line, such as previously described. Where multiple inner tubes, such as 26a and 26b, are engaged to an outer tube 28, the respective engagement areas, such as 30a and 30b, may be formed by any engagement method know, and may be the same or different. For example, in FIG. 7 both engagement areas 30a and 30b may be a weld formed through application of RF or other energy, such as IR, UV, selected frequencies of laser light, ultra-sonic and electrical energy among others. Alternatively, one engagement area, such as 30a may be such a weld, while the other engagement area, in this example 30b, is an adhesively formed bond. Any and all other configurations are encompassed by the present invention.

In the embodiment shown in FIG. 8, the inner tubes 26a and 26b, have sufficient diameters to have outer surfaces 38a and 38b respectively, which engage not only the inner surface 40 of the outer tube 28, but also each other. In such an embodiment, the tubes 26a and 26b may be provided with an inner tube engagement area 50 where they contact one another. Such an inner tube engagement area 50 may be formed and configured in the any of the manners previously discussed in terms of engagement area 30, 30a or 30b. Inner tube engagement area 50, may help to ensure orientation and stability of the inner tubes 26a and 26b not only relative to one another bu relative to the outer tube 28 as well.

In the embodiment shown in FIG. 9, a tubular member 20 is shown which includes three inner tubes 26a, 26b and 26c, within outer tube 28. Like the embodiment shown in FIG. 8, each inner tube may be engaged to the outer tube along an engagement area 30a, 30b and 30c respectively. The engagement areas may be formed in the same or different manners and may be configured in any manner desired. In the present embodiment, the inner tubes 26a, 26b and 26c, may also be provided with inner tube engagement areas 50a, 50b and 50c. The inner tube engagement areas may be formed in the same or different manners and may have any configuration desired.

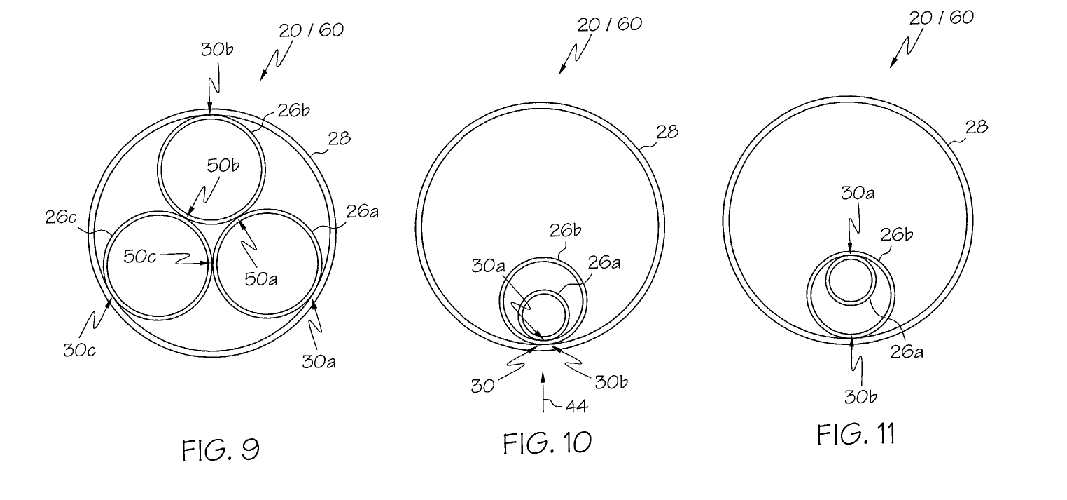

In yet other embodiments of the invention shown in FIGS. 10 and 11, a series of inner tubes 26a and 26b may be nestingly arranged within outer tube 28. In the embodiment shown in FIG. 10, the nested tubes may share a common engagement area 30 where all three nested tubes have been welded to one another through application of RF or other energy 44. Alternatively, tube 26a may be engaged to 26b along engagement area 30a, whereas tube 26b may be engaged to outer tube 28 along engagement area 30b. Where the engagement areas 30a and 30b are distinct, the particular method of formation and configuration of each area may be different or the same.

In FIG. 11, the engagement area 30a, is located at a different angular position than the engagement area 30b. The various tubes may be positioned relative to one another in any manner desired. As a result, where a tubular member 20 has more than two tubes, the engagement areas, may likewise be positioned relative to one another in any manner desired.

In all of the various embodiments shown in FIGS. 2-11, the tubular member 20, may be characterized as a catheter 60 of any type such as previously described above.

In addition to being directed to the specific combinations of features claimed below, the invention is also directed to embodiments having other combinations of the dependent features claimed below and other combinations of the features described above.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

1. A multi-lumen tubular member having a predetermined length, the tubular member comprising:
an outer tube and at least one inner tube, the outer tube substantially disposed about the at least one inner tube, the at least one inner tube having an outer surface and the outer tube having an inner surface, a portion of the outer surface of the at least one inner tube is engaged to a portion of the inner surface of the outer tube to define an engagement area, the engagement area extending in a substantially longitudinal manner along at least 10 percent of the predetermined length.

2. The tubular member of claim 1 wherein the tubular member is a catheter.

3. The tubular member of claim 1 wherein the at least one inner tube defines at least one inner lumen and the outer tube defines at least one outer lumen.

4. The tubular member of claim 1 wherein the engagement area comprises a weld between the portion of the outer surface of the at least one inner tube and the portion of the inner surface of the outer tube.

5. The tubular member of claim 4 wherein the weld is formed through the application of RF energy to the engagement area.

6. The tubular member of claim 4 wherein the weld is formed through the application of at least one form of energy selected from the group consisting of: IR energy, UV energy, selected frequencies of laser energy, ultra-sonic energy, electrical energy, and any combination thereof.

7. The tubular member of claim 1 wherein the engagement area comprises a chemical or physical bond between the portion of the outer surface of the at least one inner tube and the portion of the inner surface of the outer tube.

8. The tubular member of claim 7 wherein the chemical or physical bond is adhesively formed.

9. The tubular member of claim 1, wherein the engagement area is substantially linear.

10. The tubular member of claim 1 wherein the engagement area defines a plurality of engaged areas and unengaged areas intermittently spaced along the of the outer surface of the at least one inner tube and the inner surface of the outer tube.

11. The tubular member of claim 1 wherein the at least one inner tube is oriented in a substantially parallel manner relative to the outer tube.

12. The tubular member of claim 1 wherein the at least one inner tube is helically oriented within the outer balloon.

13. The tubular member of claim 1 wherein the at least one inner tube comprises a first inner tube and a second inner tube, a portion of the outside surface of the first inner tube being engaged at a first engagement area to a first portion of the inside surface of the outer tube, a portion of the outside surface of the second inner tube being engaged at a second engagement area to a second portion of the inside surface of the outer balloon.

14. The tubular member of claim 13 wherein at least one of the first engagement area and second engagement area comprise a weld.

15. The tubular member of claim 14 wherein the weld is formed through the application of at least one form of energy selected from the group consisting of: RF energy, IR energy, UV energy, selected frequencies of laser energy, ultra-sonic energy, electrical energy, and any combination thereof.

16. The tubular member of claim 13 wherein the at least one of the first engagement area and second engagement area comprise a chemical or physical bond.

17. The tubular member of claim 16 wherein the chemical or physical bond is adhesively formed.

18. The tubular member of claim 13, wherein the at least one of the first engagement area and the second engagement area is substantially linear.

19. The tubular member of claim 13 wherein the at least one of the first engagement area and second engagement area defines a plurality of engaged areas and unengaged areas intermittently spaced apart.

20. The tubular member of claim 13 wherein the first inner tube and the second inner tube have an inner tube engagement area, the inner tube engagement area being defined by a second portion of the outside surface of the first inner tube being in contact with a second outside portion of the outside surface of the second inner tube.

21. The tubular member of claim 1 wherein the at least one inner tube comprises at least one first inner tube disposed about a second inner tube.

22. The tubular member of claim 21 wherein the first inner tube and the second inner tube have an inner tube engagement area positioned at substantially the same angular location as the engagement area.

23. The tubular member of claim 21 wherein the first inner tube and the second inner tube have an inner tube engagement area positioned at a angular location different than that of the engagement area.

24. The tubular member of claim 1 wherein the engagement area extends along about 11 percent to about 25 percent of the predetermined length.

25. The tubular member of claim 1 wherein the engagement area extends along about 50 percent of the predetermined length.

26. The tubular member of claim 1 wherein the engagement area extends along the entire predetermined length.

27. The tubular member of claim 1 wherein the engagement area is not a web.

28. A method of making a multi-lumen tubular member comprising the following steps:
a) providing an outer tube having a predetermined length, the outer tube having an inner surface and defining an outer lumen;
b) providing at least one inner tube which defines at least one inner lumen, the at least one inner tube having an outer surface;
c) inserting the at least one inner tube into the outer lumen defined by the outer tube; and
d) engaging at least a portion of the inner surface of the outer tube to a portion of the outer surface of the at least one inner tube to form an engagement area which extends in a substantially longitudinal manner along a substantial portion of the predetermined length.

29. The method of claim 28 wherein engaging the at least a portion of the inner surface of the outer tube to a portion of the outer surface of the at least one inner tube is accomplished through the longitudinal application of at least one form of energy from the group consisting of: RF energy, IR energy, UV energy, selected frequencies of laser energy, ultra-sonic energy, electrical energy, and any combination thereof.
